(19) United States
(12) Patent Application Publication
(10) Pub. No.: US 2026/0219432 A1
(43) Pub. Date: Jul. 30, 2026

(54) OPHTHALMIC ILLUMINATION DEVICES AND METHODS FOR MANUFACTURING COUPLING ASSEMBLIES THEREOF

(71) Applicant: Alcon Inc., Fribourg (CH)

(72) Inventors: Qing Xiang, Irvine, CA (US); Alireza Mirsepassi, Irvine, CA (US)

(21) Appl. No.: 19/447,138

(22) Filed: Jan. 13, 2026

Related U.S. Application Data
(60) Provisional application No. 63/751,365, filed on Jan. 30, 2025.

(51) Int. Cl. F21V 8/00; G02B 6/255; G02B 6/38; A61B 90/30; A61F 9/007

(52) U.S. Cl. CPC G02B 6/0006; G02B 6/2551; G02B 6/2555; G02B 6/3803; A61B 90/30; A61B 2090/306; A61F 9/007

(57) ABSTRACT

Embodiments disclosed herein provide an ophthalmic illumination device. The ophthalmic illumination device includes a first optical fiber having a proximal end and a distal end, and a second optical fiber having a proximal end and a distal end. The proximal end of the first optical fiber is coupled to a light source that provides an illumination light, and the distal end of the first optical fiber is disposed within a first ferrule. The proximal end of the second optical fiber is disposed within a second ferrule, and the distal end of the second optical fiber is configured to transmit the illumination light received from the first optical fiber into an interior portion of a patient’s eye. The first ferrule is disposed against the second ferrule to couple the distal end of the first optical fiber with the proximal end of the second optical fiber.

### INTRODUCTION

Microsurgical procedures frequently involve precision sealing, cutting, and/or removing of various body tissues. For example, certain ophthalmic surgical procedures may involve sealing, cutting, and/or removing tissues within the posterior segment of the eye. During such procedures, an illumination device may be used to help the surgeon illuminate and visualize an area of treatment within the posterior segment of the eye. Therefore, illumination devices need to provide a sufficient amount of light to ensure the area of treatment is adequately illuminated.

Typically, a first optical fiber transmits light that is generated by a light source in a surgical console to the illumination device, and a second optical fiber receives the light propagated by the first optical fiber and transmits it into an eye. However, in certain existing implementations, poor coupling between the optical fibers results in loss of light during transmission within the illumination device. Consequently, the illumination device may not provide adequate illumination within the eye, thereby restricting the surgeon’s visualization of the area of treatment, which limits efficiency and can make it difficult to operate on the patient’s eye and potentially lead to unwanted and unintentional trauma to ocular tissues.

### BRIEF SUMMARY

The present disclosure relates generally to ophthalmic illumination devices and methods for manufacturing coupling assemblies thereof.

In certain embodiments, an ophthalmic illumination device is provided. The ophthalmic illumination device includes a first optical fiber having a proximal end and a distal end, and a second optical fiber having a proximal end and a distal end. The proximal end of the first optical fiber is coupled to a light source that provides an illumination light, and the distal end of the first optical fiber is disposed within a first ferrule. The proximal end of the second optical fiber is disposed within a second ferrule, and the distal end of the second optical fiber is configured to transmit the illumination light received from the first optical fiber into an interior portion of a patient’s eye. The first ferrule is disposed against the second ferrule to couple the distal end of the first optical fiber with the proximal end of the second optical fiber.

In certain embodiments, a method for manufacturing a coupling assembly of an ophthalmic illumination device is provided. The method includes inserting a first optical fiber into a first ferrule and a second optical fiber into a second ferrule, heating a distal end of the first optical fiber and a proximal end of the second optical fiber to expand the distal end of the first optical fiber within the first ferrule and the proximal end of the second optical fiber within the second ferrule, inserting the first ferrule and the second ferrule into an alignment sleeve, and applying an adhesive through a slit in the alignment sleeve to couple the distal end of the first optical fiber with the proximal end of the second optical fiber, where the second optical fiber is configured to transmit an illumination light received from the first optical fiber into an interior portion of a patient’s eye.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1A shows an example ophthalmic surgical system that may be used to perform ophthalmic procedures on an eye, according to certain embodiments.

FIG. 1B shows example components of a surgical console of the ophthalmic surgical system shown in FIG. 1A, according to certain embodiments.

FIG. 2 shows an example of an illumination device that is coupled to a light source of the ophthalmic surgical system shown in FIGS. 1A-1B through a cable and a connector, according to certain embodiments.

FIG. 3A shows a side view of the illumination device of FIG. 2, according to certain embodiments.

FIG. 3B shows a cross-sectional side view of the illumination device of FIG. 3A, according to certain embodiments.

FIG. 3C shows a cross-sectional side view of a coupling assembly within the illumination device of FIG. 3B, according to certain embodiments.

FIG. 3D shows a perspective view of the coupling assembly of FIG. 3C, according to certain embodiments.

FIG. 4 illustrates a cross-sectional side view of an eye with the illumination device of FIGS. 1A-1B, according to certain embodiments.

FIG. 5 shows a flowchart of a method for manufacturing an illumination device with a coupling assembly, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

Reference throughout this specification to the term “distal” refers to a system, device, component, end, portion, or segment that is disposed closer to a patient and/or further from a console during an ophthalmic procedure; and the term “proximal” refers to the system, device, component, end, portion, or segment that is disposed further from the patient and/or closer to the console during the ophthalmic procedure.

During ophthalmic surgeries, illumination devices (e.g., chandeliers, endoilluminators, wide angle illuminators, illumination laser probes, etc.) are often used to illuminate a treatment area and/or target tissue within an eye. The illumination light transmitted by the illumination device may be used to help provide illumination while another tool (e.g., a vitrectomy probe, a diathermy probe, forceps, etc.) is used to operate on the treatment area or target tissue. Therefore, the illumination device may need to provide sufficient illumination while operating on the target tissue so that the surgeon is able to adequately see the target tissue, move between different treatment areas, and avoid accidentally or unintentionally causing damage to other surrounding tissues.

Current illumination devices often include a larger, proximal optical fiber that is coupled with a smaller, distal optical fiber within a handpiece of the illumination device. The proximal fiber is connected to a light source within a surgical console and propagates light received from the light source to the distal optical fiber, which then emits the light from the illumination device. In particular, the larger proximal optical fiber is used to aid propagation from the light source to the illumination device, and the smaller distal optical fiber is used to avoid unnecessarily large incisions in an eye. To ensure adequate transmission of illumination light from the proximal optical fiber to the distal optical fiber, a proximal end of the distal optical fiber may be expanded (e.g., by applying heat to the proximal end) to help match a diameter or mode size of the proximal end to a diameter or mode size of the larger proximal optical fiber. Once the proximal end of the distal optical fiber is expanded, the distal optical fiber is coupled with the proximal optical fiber using a coupling component.

The embodiments described herein may improve the coupling efficiency of the coupling assemblies for illumination devices. For example, a coupling assembly as described herein allows illumination light to be transmitted from the proximal optical fiber to the distal optical fiber with minimal light loss, thereby improving the effectiveness of light transmitted by illumination devices and providing the surgeon with greater visualization of the treatment area or target tissue. The coupling assemblies described herein, therefore, provide illumination devices with greater light transmission efficiency, which thereby facilitates safer, quicker, and more efficient ophthalmic procedures.

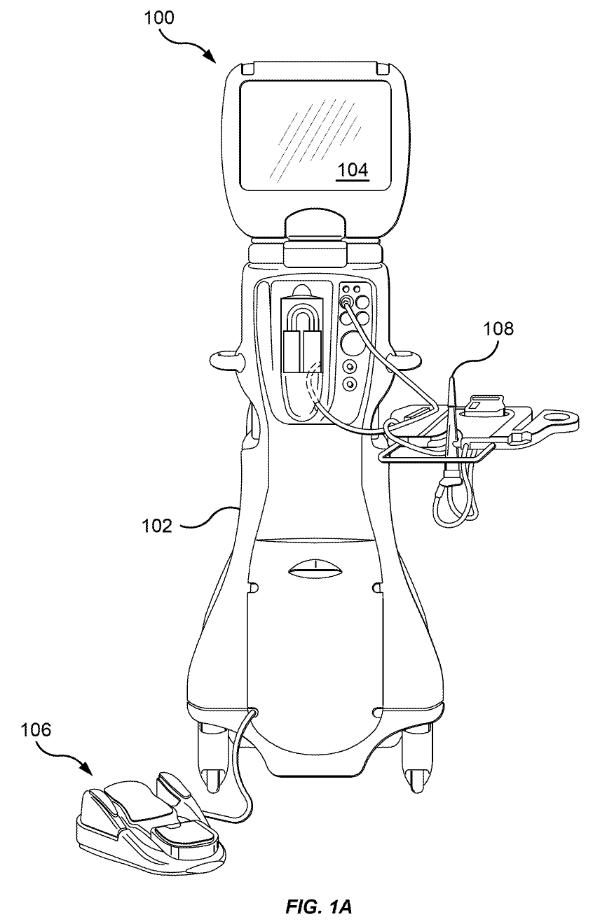

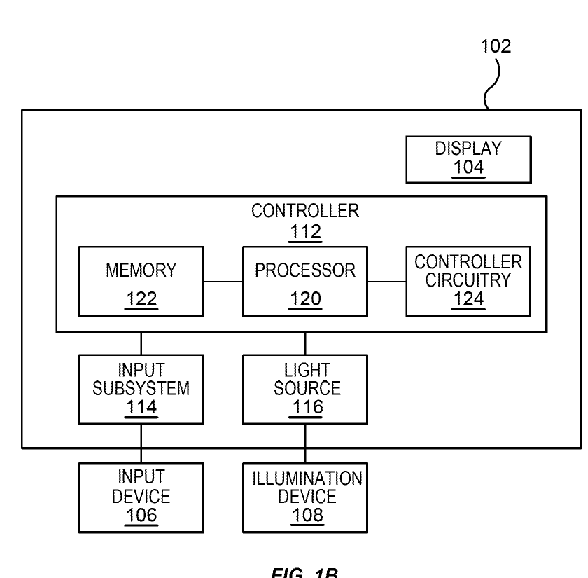

FIG. 1A shows an example ophthalmic surgical system 100 that may be used to perform ophthalmic procedures on an eye, according to certain embodiments. The ophthalmic surgical system 100 includes a console 102 (also referred to as a “surgical console”), which includes a display 104, an input device 106 (e.g., a foot pedal), and an illumination device 108 (also referred to as an “illumination handpiece,” an “illumination instrument,” or an “illumination probe”). The components of the ophthalmic surgical system 100 and the surgical console 102 are mechanically and/or electrically coupled as shown and described in more detail with reference to FIG. 1B.

FIG. 1B shows example components of the surgical console 102 of the ophthalmic surgical system 100 shown in FIG. 1A, according to certain embodiments. As shown, the surgical console 102 includes a controller 112, an input subsystem 114, a light source 116, and a display 104. The controller 112 controls the operation of the surgical console 102 and is illustrated as being operationally coupled to the input device 106 via input subsystem 114, and to the illumination device 108 via the light source 116. The controller 112 includes a processor 120, a memory 122, and controller circuitry 124.

The processor 120 may be any type of general purpose processor or could be a processor specifically designed for driving the input subsystem 114 and light source 116 illustrated in FIG. 1B, such as an application-specific integrated circuit (“ASIC”). The processor 120 may be, or include, a microprocessor, a microcontroller, an embedded microcontroller, a programmable digital signal processor, or any other programmable device operable to execute instructions stored in the memory 122 for operating the surgical console 102. For example, the processor 120 may execute instructions in the memory 122 to receive inputs provided by the input device 106 through the input subsystem 114 and, in response, send instructions to the light source 116 for controlling light transmitted by the illumination device 108. Further, the processor 120 may execute instructions to generate user interface view for display by the display 104. In some instances, the processor 120 may also be or include a programmable gate array, programmable array logic, or any other device of combinations of devices operable to process electric signals.

The memory 122 can be any type of storage device or non-transitory computer-readable medium, such as random-access memory (“RAM”) or read-only memory (“ROM”), which is operable to receive, store, or recall data, including, but not limited to, electronic, magnetic, or optical memory, whether volatile or non-volatile. The memory 122 stores instructions executed by the processor 120. In example embodiments, functionality disclosed herein can be provided by the processor 120 and the memory 122 (i.e., software based), by the controller circuitry 124 (i.e., hardware based), or by a combination thereof. The memory 122 may include code stored thereon. The code may include instructions that may be executable by the processor 120. The code may be created, for example, using any programming language, including but not limited to, C, C++, Java, Python, Rust, or any other programming language (including assembly languages, hardware description languages, and database programming languages). In some instances, the code may be a program that, when executed by the processor 120, causes the surgical console 102 to operate input subsystem 114 and/or light source 116 for, e.g., driving the illumination device 108 or other devices in communication with the surgical console 102.

The illumination device 108 may be any suitable ophthalmic illumination instrument that can be operated on the basis of the embodiments described herein. For example, the illumination device 108 may be an endoilluminator, wide angle illuminator, chandelier, illumination laser probe, etc.

As shown, the light source 116 is in communication with the controller 112 and is configured to propagate illumination light for transmission to the illumination device 108. In operation, upon receiving a control signal from the controller 112, the light source 116 produces the illumination light. Further, the controller 112 is configured to control the settings of the light source 116 based, for example, on user input. For example, a color and/or brightness of the illumination light can be adjusted by the surgeon (e.g., using the surgical console 102 and/or the foot controller 110) by controlling one or more settings of the light source 116. In certain embodiments, the light source 116 can generate light with various levels of brightness. For example, in certain embodiments, the light source 116 can generate illumination light with up to 10 lumens (e.g., up to 9 lumens, 8 lumens, 7 lumens, 6 lumens, or 5 lumens) and, in certain other embodiments, more than 10 lumens.

In another example, the light source 116 can generate light with one of a variety of colors. For example, the light source 116 may comprise one or more light-emitting diodes (LEDs) and, therefore, the illumination light may be the product of any combination of one or more of a red light, a green light, or a blue light. In particular, two or more of the red, green, and blue lights can be combined to produce a wide spectrum of colors, e.g., yellow, magenta, cyan, white, etc.

The input device 106 may be any device that is capable of receiving commands from the user of the surgical console 102 in order to operate the illumination device 108 and/or other components of the surgical console 102. In FIG. 1A, the input device 106 is illustrated as a foot pedal, however, other types of input devices are also within the scope of the disclosure. In one example, the user provides a command to the input device 106, which is received and relayed to the controller 112 by the input subsystem 114. In response, the controller 112 sends instructions to the light source 116 to control the operations of the illumination device 108 based on the user command. As such, the surgical console 102 and/or the foot controller 110 are configured to control the illumination device 108 and operational features thereof.

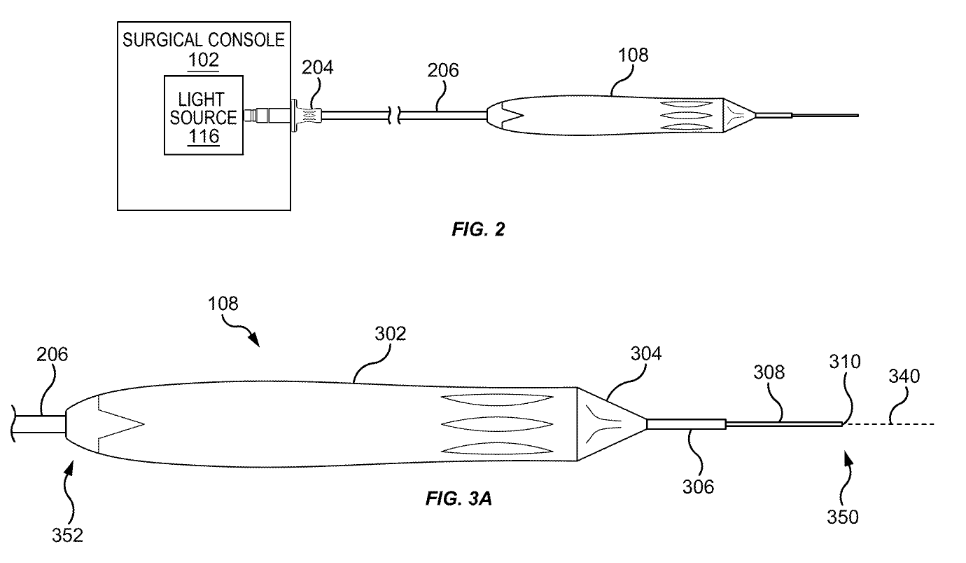

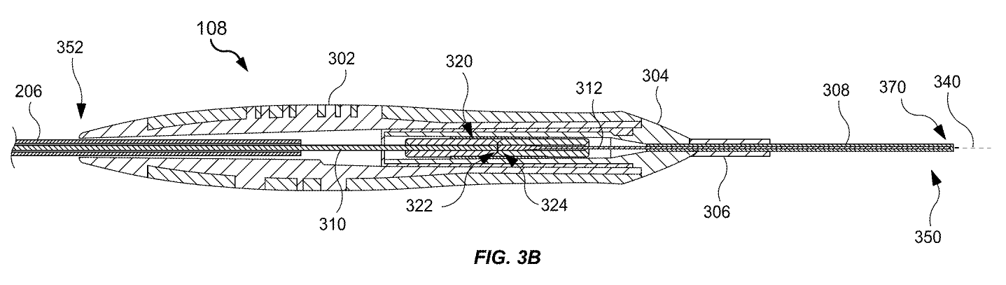

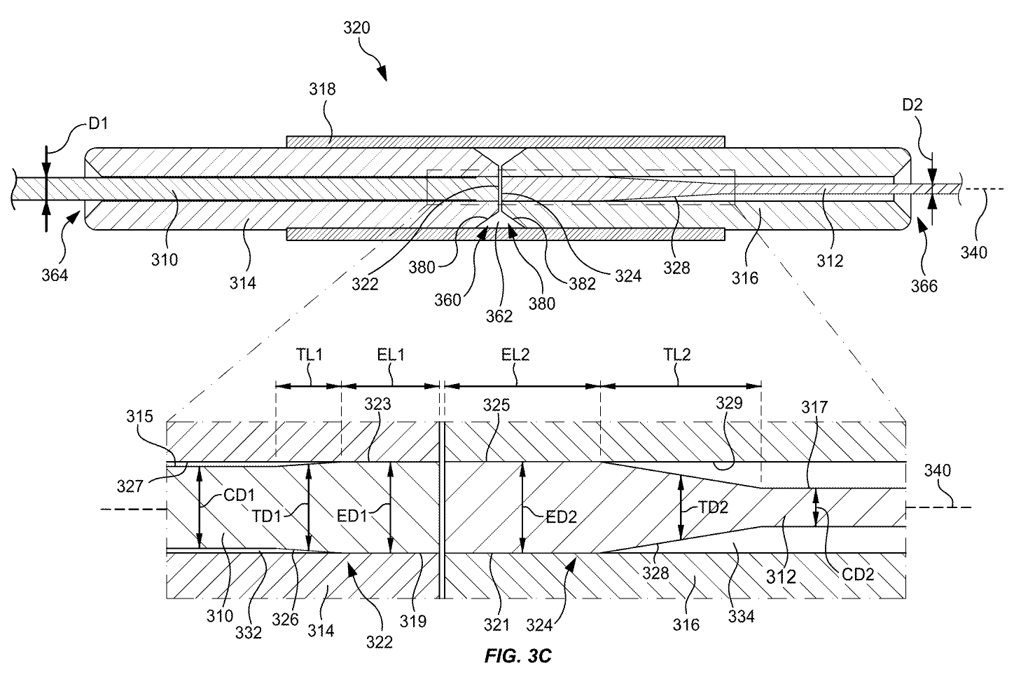

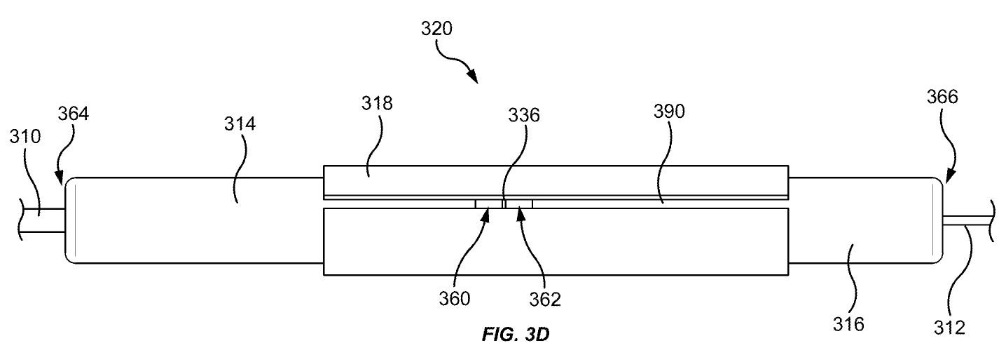

FIG. 2 shows an example of the illumination device 108 that is coupled to the light source 116 shown in FIGS. 1A-1B through a cable 206 and a connector 204, according to certain embodiments. In particular, the connector 204 is coupled to the illumination device 108 via the cable 206, which includes an optical fiber (e.g., optical fiber 310 seen in FIG. 3B) disposed therein. The connector 204 is coupled to the light source 116, for example, via a port at the surgical console 102. In certain embodiments, the illumination light produced by the light source 116 is condensed and focused on an opening exposing a proximal end of the optical fiber extending through the cable 206. In certain embodiments, the opening at the proximal end of the optical fiber is disposed within the connector 204.

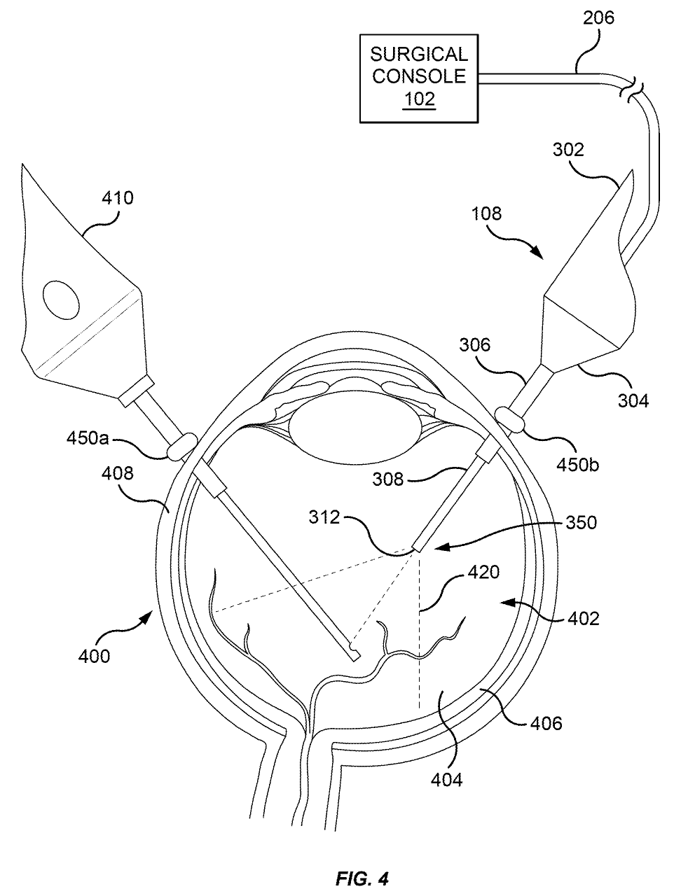

The illumination device 108 is then operable to transmit the illumination light received from the light source 116 such that the illumination light is propagated along the cable 206 and through the illumination device 108 by the optical fiber disposed therein. The illumination light transmitted by the illumination device 108 may be used, for example, to illuminate a posterior segment (or interior portion) of an eye to assist a surgeon in ophthalmic procedures as described in further detail with reference to FIG. 4.

FIG. 3A shows a side view of the illumination device 108 of FIG. 2, according to certain embodiments. FIG. 3B shows a cross-sectional side view of the illumination device 108 of FIG. 3A, according to certain embodiments. Accordingly, FIGS. 3A-3B are described together herein for clarity purposes.

The illumination device 108 includes a handpiece 302 with a handpiece tip 304. In certain embodiments, the handpiece 302 is configured to be held by a user, such as a surgeon. For example, the handpiece 302 may be ergonomically contoured to substantially fit the hand of the user. In certain embodiments, the outer surface may be textured or have one or more gripping features formed thereon, such as one or more grooves and/or ridges. The handpiece 302 may be made from any materials commonly used for such instruments and suitable for ophthalmic surgery. For example, the handpiece 302 may be formed of a lightweight aluminum, a polymer, or other suitable material. In some embodiments, the handpiece 302 may be sterilized and used in more than one surgical procedure, or may be a single-use device.

The illumination device 108 further includes a protective sleeve 306 and a needle 308 extending from the handpiece tip 304 towards a distal end 350 of the illumination device 108. The needle 308 is disposed within the protective sleeve 306 and extends along a longitudinal axis 340 of the illumination device 108. The cable 206 is coupled to a proximal end 352 of the illumination device 108.

Turning to FIG. 3B, the illumination device 108 further includes a first optical fiber (or proximal optical fiber) 310 and a second optical fiber (or distal optical fiber) 312. The first optical fiber 310 runs through a cable 206 to optically couple the light source 116 to the handpiece 302. For example, a proximal end of the first optical fiber 310 may be disposed within the connector 204 shown in FIG. 2 and a distal end 322 of the first optical fiber 310 is disposed within the handpiece 302. The second optical fiber 312 is disposed within the handpiece 302 and the needle 308. That is, a proximal end 324 of the second optical fiber 312 is disposed within the handpiece 302 and a distal end 370 of the second optical fiber 312 is disposed within the needle 308 near the distal end 350 of the illumination device 108. The first optical fiber 310 and the second optical fiber 312 are coupled within a coupling assembly 320 as described in further detail with reference to FIG. 3C.

In certain embodiments, the light source 116 focuses (or guides) the illumination light onto the proximal end of the first optical fiber 310 via the connector 204. The illumination light then travels through the first optical fiber 310, which extends from the connector 204 into the handpiece 302, and is transmitted from the distal end (or emitting end) 322 of the first optical fiber 310 onto the proximal end 324 of the second optical fiber 312. As such, the illumination light then travels through the second optical fiber 312 and is transmitted from the distal end (or emitting end) 370 of the second optical fiber 312 through the needle 308.

In certain embodiments, the needle 308 is comprised of stainless steel, but may include a window or other transparent protective element at the distal end 370 of the second optical fiber 312 and within the needle 308. In certain embodiments, the first optical fiber 310 and/or the second optical fiber 312 are comprised of a plastic or polymer-based fiber, but may also be comprised of other commonly used optical fiber materials or other suitable materials for transmitting the illumination light.

FIG. 3C shows a cross-sectional side view of the coupling assembly 320 within the illumination device 108 of FIG. 3B, according to certain embodiments. FIG. 3D shows a perspective view of the coupling assembly 320 of FIG. 3C, according to certain embodiments. Accordingly, FIGS. 3C-3D are described together herein for clarity purposes.

The coupling assembly 320 shown in FIGS. 3C-3D further includes a first ferrule (or proximal ferrule) 314, a second ferrule (or distal ferrule) 316, and an alignment sleeve 318. The first ferrule 314 comprises a first channel (or proximal channel) 332 and a distal angled surface 380 at a distal end 360 of the first ferrule 314. The second ferrule 316 comprises a second channel (or distal channel) 334 and a proximal angled surface 382 at a proximal end 362 of the second ferrule 316. The distal angled surface 380 and the proximal angled surface 382 form a cavity 336 within the alignment sleeve 318, which is accessible via a slit 390 in the alignment sleeve 318. In certain embodiments, an adhesive (e.g., an epoxy or glue having a matching, or substantially similar refractive index to the optical fibers) is applied to the cavity 336 via the slit 390 to couple the first ferrule 314 with the second ferrule 316. The first ferrule 314 and the second ferrule 316 are coupled in an end-to-end (or butt-to-butt) configuration, such that the distal end 360 of the first ferrule 314 is in complete (or almost complete considering the adhesive) contact with the proximal end 362 of the second ferrule 316. In other words, the first ferrule 314 is disposed against the second ferrule 316 within the alignment sleeve 318.

In certain embodiments, the first ferrule 314 and the second ferrule 316 are comprised of one or more of a ceramic material, a metallic material (e.g., stainless steel), or other similar material. In certain embodiments, the alignment sleeve 318 is comprised of a ceramic material and is configured to radially expand (or bend) upon insertion of the ferrules 314, 316 therein, such that the alignment sleeve 318 functions as a clamp having inward forces that help hold the ferrules 314, 316 in place within the alignment sleeve 318.

As shown in FIG. 3C, the first optical fiber 310 is at least partially disposed within the first channel 332 of the first ferrule 314, and the second optical fiber 312 is at least partially disposed within the second channel 334 of the second ferrule 316. That is, the distal end 322 of the first optical fiber 310 is disposed within the first ferrule 314, and the proximal end 324 of the second optical fiber 312 is disposed within the second ferrule 316. The distal end 322 of the first optical fiber 310 is completely (or almost entirely) flush with the distal end 360 of the first ferrule 314, and the proximal end 324 of the second optical fiber 312 is completely (or almost entirely) flush with the proximal end 362 of the second ferrule 316. Thus, the first optical fiber 310 and the second optical fiber 312 are coupled in an end-to-end (or butt-to-butt) configuration, similar to the first ferrule 314 and the second ferrule 316.

The distal end 322 of the first optical fiber 310 comprises an end portion (or first end portion) 319 and a tapered portion (or first tapered portion) 326. The end portion 319 of the first optical fiber 310 has a constant, or at least a substantially constant, diameter (ED1) along a length (EL1), and the tapered portion 326 has a diameter (TD1) that tapers towards a core portion 315 of the first optical fiber 310 along a length (TL1). In other words, the tapered portion 326 tapers towards the proximal end of the first optical fiber 310. The core portion 315 has a diameter (CD1) that corresponds to an original diameter or mode size of the first optical fiber 310. The diameters (ED1 and TD1) of the end portion 319 and the tapered portion 326 are larger than the diameter (CD1) of the core portion 315 due to heat being applied to the distal end 322 of the first optical fiber 310, causing the expansion thereof.

The proximal end 324 of the second optical fiber 312 also comprises an end portion (or second end portion) 321 and a tapered portion (or second tapered portion) 328. The end portion 321 of the second optical fiber 310 has a constant, or at least a substantially constant, diameter (ED2) along a length (EL2), and the tapered portion 328 has a diameter (TD2) that tapers towards a core portion 317 of the second optical fiber 312 along a length (TL2). In other words, the tapered portion 328 tapers towards the distal end of the second optical fiber 312. The core portion 317 has a diameter (CD2) that corresponds to an original diameter or mode size of the second optical fiber 312. The diameters (ED2 and TD2) of the end portion 321 and the tapered portion 328 are larger than the diameter (CD2) of the core portion 317 due to heat being applied to the proximal end 324 of the second optical fiber 312, causing the expansion thereof. In certain embodiments, the size of the diameter (ED2) of the second optical fiber 312 matches the size of the diameter (ED1) of the first optical fiber 310.

In certain embodiments, ED1 and/or ED2 are between 250 micrometers (µm) and 450 µm (e.g., between 260 µm and 440 µm, 270 µm and 430 µm, or 280 µm and 420 µm). In certain embodiments, CD1 is between 250 µm and 450 µm (e.g., between 260 µm and 440 µm, 270 µm and 430 µm, or 280 µm and 420 µm) and CD2 is between 50 µm and 350 µm (e.g., between 60 µm and 340 µm, 70 µm and 330 µm, or 80 µm and 320 µm). In certain embodiments, ED2 may be greater than or equal to ED1.

As shown in FIG. 3C, the diameter (TD1) of the tapered portion 326 decreases in size from the end portion 319 to the core portion 315 of the first optical fiber 310, and the diameter (TD2) of the tapered portion 328 decreases in size from the end portion 321 to the core portion 317 of the second optical fiber 312. In certain embodiments, the tapered portion 326 of the first optical fiber 310 is uniform (or straight) along the length (TL1) of the tapered portion 326, and the tapered portion 328 of the second optical fiber 312 is uniform (or straight) along the length (TL2) of the tapered portion 328. As an example, a “uniform” taper implies that the diameters (TD1 and TD2) of the tapered portions 326 and 328 equally decrease in size relative to the longitudinal axis 340 along the corresponding lengths thereof, i.e., TL1 and TL2. In other words, the tapered portions 326 and 328 are symmetrical relative to and along the longitudinal axis 340 of the illumination device 108 and/or the longitudinal axes of the first optical fiber 310 and the second optical fiber 312, respectively.

In certain embodiments, to form the tapered portions 326 and 328, the first optical fiber 310 is inserted into the first channel 332 through a proximal end 364 of the first ferrule 314, and the second optical fiber 312 is inserted into the second channel 334 through a distal end 366 of the second ferrule 316. Before the ferrules 314, 316 are inserted into the alignment sleeve 318, heat is applied to the distal end 322 of the first optical fiber 310 and the proximal end 324 of the second optical fiber 312. By heating the first optical fiber 310 and the second optical fiber 312, the distal end 322 of the first optical fiber 310 and the proximal end 324 of the second optical fiber 312 expand within the first ferrule 314 and the second ferrule 316, respectively, thereby forming the tapered portions 326 and 328.

An amount of time and/or a temperature at which the optical fibers 310, 312 are heated can be adjusted to achieve desired shapes of the end portions 319, 321 and the tapered portions 326, 328. In certain embodiments, the second optical fiber 312 may be heated for a longer amount of time and/or at greater temperature than the first optical fiber 310, which causes the lengths (EL2 and TL2) of the end portion 321 and the tapered portion 328 to be greater than the lengths (EL1 and TL1) of the end portion 319 and the tapered portion 326. For example, the first optical fiber 310 and the second optical fiber 312 are both heated at 550 °F (degrees Fahrenheit), but the distal end 322 of the first optical fiber 310 is heated for 2-3 seconds and the proximal end 324 of the second optical fiber 312 is heated for 8-10 seconds. In certain embodiments, the lengths (EL2 and TL2) of the end portion 321 and the tapered portion 328 are greater than the lengths (EL1 and TL1) of the end portion 319 and the tapered portion 326 to provide gradual compression of the illumination light as it travels distally towards the distal end of the second optical fiber 312.

As shown in FIG. 3C, the distal end 322 of the first optical fiber 310 and the proximal end 324 of the second optical fiber 312 expand to fill the first channel 332 of the first ferrule 314 and the second channel 334 of the second ferrule 316, respectively, along the end portions 319, 321. As such, the expansion reduces lateral mismatch between the optical fibers 310, 312 and centralizes the optical fibers 310, 312 within the channels 332, 334 along the longitudinal axis 340. The expansion ratio ED2/CD2 of the second optical fiber 312 may be greater than the expansion ratio ED1/CD1 of the first optical fiber 310. In certain embodiments, the expansion of the second optical fiber 312 allows a ratio between the diameters ED2 and CD2 of the end portion and core portions 317 of the second optical fiber 312 to be greater than, for example, two (or more) because the diameter (ED2) at the end portion 321 of the second optical fiber 312 and the diameter (ED1) at the end portion 319 of the first optical fiber 310 are expanded to match each other. In other words, a wide range of different sized optical fibers can be coupled by expanding the corresponding distal and proximal ends thereof.

Further, expanding the optical fibers 310, 312 to fill the channels 332, 334 eliminates gaps (or at least substantially eliminates the gap) between an outer surface 323 of the first optical fiber 310’s distal end 322 and an inner surface 327 of the first channel 332, and between an outer surface 325 of the second optical fiber 312’s proximal end 324 and an inner surface 329 of the second channel 334. In certain embodiments, the expansion of the optical fibers 310, 312 within the channels 332, 334 forms a mechanical lock that holds the optical fibers 310, 312 in place inside the ferrules 314, 316. In certain embodiments, the mechanical lock eliminates the need for an adhesive to hold the optical fibers 310, 312 within the ferrules 314, 316. However, it is also contemplated that, in certain embodiments, an adhesive may be used to help hold the optical fibers 310, 312 inside the ferrules 314, 316.

By coupling the tapered portion 326 of the first optical fiber 310 with the tapered portion 328 of the second optical fiber 312 in the end-to-end configuration shown in FIG. 3C, the coupling assembly 320 provides an improved coupling efficiency between the first optical fiber 310 and the second optical fiber 312 relative to couplings between two fibers in existing illumination devices. For example, the expanded distal end 322 of the first optical fiber 310 and the expanded proximal end 324 of the second optical fiber 312 allow for a larger effective coupling area, making it easier to align the illumination light transmitted by the first optical fiber 310 with the second optical fiber 312. In other words, the expanded proximal end 324 of the second optical fiber 312 is able to easily capture the illumination light received from the distal end 322 of the first optical fiber 310. As a result of the improved alignment between the two optical fibers 310, 312, the coupling assembly 320 provides an improved alignment tolerance, allows for adiabatic transition of the illumination light, and minimizes light loss due to misalignment.

Once the illumination light is received at the proximal end 324 of the second optical fiber 312, the tapered portion 328 of the second optical fiber 312 gradually guides and compresses the illumination light as it travels distally towards the distal end of the second optical fiber 312. In other words, the smooth transition in size, or uniform shape of the tapered portion 328 maintains the integrity of the illumination light as it propagates from the proximal end 324 to the distal end of the second optical fiber 312. Thus, the illumination device 108 is able to efficiently transmit the illumination light without substantial loss of light between the first optical fiber 310 and the second optical fiber 312, and/or by the second optical fiber 312.

FIG. 4 illustrates a cross-sectional side view of an eye 400 with the illumination device 108 of FIGS. 1A-1B, according to certain embodiments. The eye 400 includes a vitreous cavity 402 with vitreous 404, a retina 406, and a sclera 408. The surgical console 102 and the illumination device 108 are connected to each other via the cable 206.

In the example of FIG. 4, the illumination device 108 and a vitrectomy probe 410, are inserted into the eye 400. In particular, the vitrectomy probe 410 is inserted through a first cannula 450a and the illumination device 108 is inserted through a second cannula 450b. The illumination device 108 is configured to transmit an illumination light 420 into the vitreous cavity 402 of the eye 400 via the second optical fiber 312, e.g., based on one or more control signals received from the surgical console 102. As an example, the second optical fiber 312 is configured to transmit the illumination light 420 with a particular brightness, color, etc. The illumination light 420 thereby illuminates the vitreous cavity 402.

Although FIG. 4 shows the illumination device 108 being used with the vitrectomy probe 410, the illumination device 108 may also be used with other ophthalmic surgical instruments (e.g., a diathermy probe, forceps, pics, etc.).

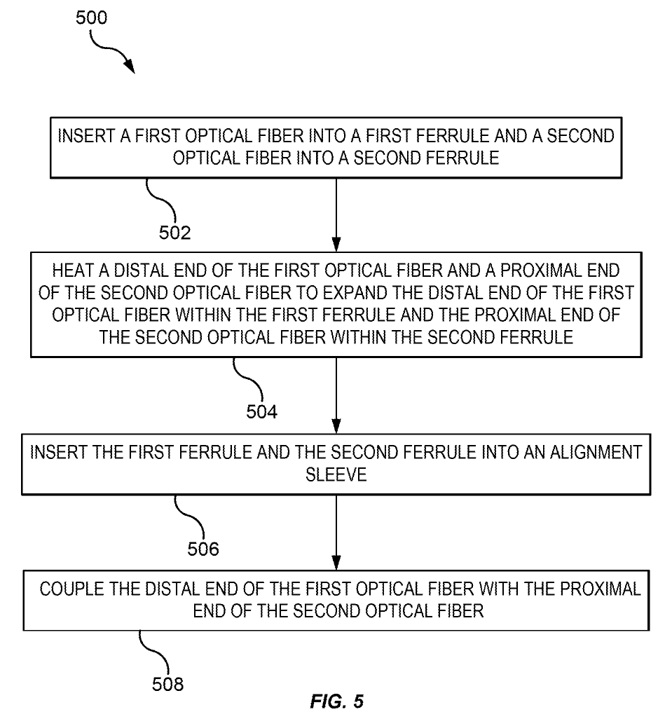

FIG. 5 shows a flowchart of a method 500 for manufacturing an illumination device with a coupling assembly, such as the coupling assembly 320 of FIG. 3C, according to certain embodiments.

At block 502, the method 500 includes inserting the first optical fiber 310 into the first ferrule 314 and the second optical fiber 312 into the second ferrule 316. In some embodiments, the first optical fiber 310 and the second optical fiber 312 are inserted such that the distal end 322 of the first optical fiber 310 is flush with the distal end 360 of the first ferrule 314, and the proximal end 324 of the second optical fiber 312 is flush with the proximal end 362 of the second ferrule 316.

At block 504, the method 500 includes heating the distal end 322 of the first optical fiber 310 and the proximal end 324 of the second optical fiber 312 to expand the distal end 322 of the first optical fiber 310 within the first ferrule 314 and the proximal end 324 of the second optical fiber 312 within the second ferrule 316. In some embodiments, a fiber beller is used to heat the first optical fiber 310 and the second optical fiber 312. As an example, the fiber beller operates at a temperature that is between 400 °F and 700 °F (e.g., between 410 °F and 690 °F, 420 °F and 680 °F, or 430 °F and 670 °F). In certain embodiments, the amount of time and/or temperature at which the first optical fiber 310 and the second optical fiber 312 are heated can be adjusted to achieve a desired shape of the tapered portions 326 and 328.

Further, in certain embodiments, during the heating at block 504, the optical fibers 310, 312 may be pulled through the corresponding ferrules 314, 316. The optical fibers 310, 312 are then removed from the beller and any excess length of the distal end 322 of the first optical fiber 310 and the proximal end 324 of the second optical fiber 312 (e.g., extending from the coupling ends of the ferrules 314, 316) may be cut to be flush with the coupling ends of the ferrules 314, 316. The coupling ends of the ferrules 314, 316 (e.g., the distal end 360 of the first ferrule 314 and the proximal end 362 of the second ferrule 316) may then be polished and/or gently wiped with a polishing film.

At block 506, the method 500 includes inserting the first ferrule 314 and the second ferrule 316 into the alignment sleeve 318. In some embodiments, the alignment sleeve 318 expands radially along the longitudinal axis 340 when the first ferrule 314 and the second ferrule 316 are inserted into the alignment sleeve 318. In some embodiments, the first ferrule 314 and the second ferrule 316 are inserted into the alignment sleeve 318 such that the first ferrule 314 is disposed against the second ferrule 316 in an end-to-end configuration.

At block 508, the method 500 includes coupling the distal end 322 of the first optical fiber 310 with the proximal end 324 of the second optical fiber 312. For example, the distal end 322 of the first optical fiber 310 can be coupled with the proximal end 324 of the second optical fiber 312 by applying an adhesive through the slit 390 in the alignment sleeve 318. In some embodiments, the adhesive is applied to fill the cavity 336 formed by the first ferrule 314, the second ferrule 316, and the alignment sleeve 318. As an example, the adhesive may include glue, epoxy, or other suitable adhesive for coupling the first ferrule 314 with the second ferrule 316.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein but are to be accorded the full scope consistent with the language of the claims.

What is claimed is:

1. An ophthalmic illumination device comprising:
a first optical fiber having a proximal end and a distal end, wherein:
the proximal end of the first optical fiber is configured to couple to a light source that provides an illumination light; and
the distal end of the first optical fiber is disposed within a first ferrule; and
a second optical fiber having a proximal end and a distal end, wherein:
the proximal end of the second optical fiber is disposed within a second ferrule; and
the distal end of the second optical fiber is configured to transmit the illumination light received from the first optical fiber into an interior portion of a patient’s eye;
wherein the first ferrule is disposed against the second ferrule to couple the distal end of the first optical fiber with the proximal end of the second optical fiber.

2. The ophthalmic illumination device of claim 1, wherein the proximal end of the second optical fiber comprises a tapered portion that tapers towards the distal end of the second optical fiber.

3. The ophthalmic illumination device of claim 2, wherein the tapered portion of the second optical fiber is symmetrical relative to and along a longitudinal axis of the second optical fiber.

4. The ophthalmic illumination device of claim 1, wherein the distal end of the first optical fiber comprises a tapered portion that tapers towards the proximal end of the first optical fiber.

5. The ophthalmic illumination device of claim 4, wherein the tapered portion of the first optical fiber is symmetrical relative to and along a longitudinal axis of the first optical fiber.

6. The ophthalmic illumination device of claim 1, wherein:
the distal end of the first optical fiber comprises:
a first end portion having a first constant diameter along a first length, and
a first tapered portion having a first diameter that tapers from the first end portion towards the proximal end of the first optical fiber; and
the proximal end of the second optical fiber comprises:
a second end portion having a second constant diameter along a second length, and
a second tapered portion having a second diameter that tapers from the second end portion towards the distal end of the second optical fiber.

7. The ophthalmic illumination device of claim 6, wherein the second end portion and the second tapered portion each have corresponding lengths that are greater than corresponding lengths of each of the first end portion and the first tapered portion.

8. The ophthalmic illumination device of claim 6, wherein a size of the first constant diameter is smaller than or equal to a size of the second constant diameter.

9. The ophthalmic illumination device of claim 1, wherein the first ferrule and the second ferrule are comprised of a ceramic material or a metallic material.

10. The ophthalmic illumination device of claim 1, further comprising:
an alignment sleeve,
wherein the first ferrule and the second ferrule are at least partially disposed within the alignment sleeve.

11. The ophthalmic illumination device of claim 10, wherein the alignment sleeve comprises a slit through which an adhesive is applied to couple the first ferrule with the second ferrule.

12. A method for manufacturing a coupling assembly of an ophthalmic illumination device comprising:
inserting a first optical fiber into a first ferrule and a second optical fiber into a second ferrule;
heating a distal end of the first optical fiber and a proximal end of the second optical fiber to expand the distal end of the first optical fiber within the first ferrule and the proximal end of the second optical fiber within the second ferrule;
inserting the first ferrule and the second ferrule into an alignment sleeve; and
applying an adhesive through a slit in the alignment sleeve to couple the distal end of the first optical fiber with the proximal end of the second optical fiber;
wherein the second optical fiber is configured to transmit an illumination light received from the first optical fiber into an interior portion of a patient’s eye.

13. The method of claim 12, wherein heating the distal end of the first optical fiber and the proximal end of the second optical fiber forms:
a tapered portion at the distal end of the first optical fiber; and
a tapered portion at the proximal end of the second optical fiber.

14. The method of claim 13, wherein desired shapes of the tapered portion of the first optical fiber and the tapered portion of the second optical fiber are achieved by adjusting an amount of time or temperature at which the first optical fiber and the second optical fiber are heated.

15. The method of claim 12, wherein heating the distal end of the first optical fiber and the proximal end of the second optical fiber causes:
a diameter of the distal end of the first optical fiber to expand within the first ferrule; and
a diameter of the proximal end of the second optical fiber to expand within the second ferrule.
